(19) United States
(12) Patent Application Publication
(10) Pub. No.: US 2025/0080931 A1
(43) Pub. Date: Mar. 6, 2025

(54) SYSTEMS, METHODS, AND DEVICES FOR ACOUSTICALLY ENHANCING IMPLANTS

(71) Applicant: GS-HealthMatrix, LLC, Los Angeles, CA (US)

(72) Inventors: Joseph Schwab, Los Angeles, CA (US); Amirhossein Yazdkhasti, Los Angeles, CA (US); Hamid Ghaednia, Los Angeles, CA (US)

(73) Assignee: GS-HealthMatrix, LLC, Los Angeles, CA (US)

(21) Appl. No.: 18/818,990

(22) Filed: Aug. 29, 2024

Related U.S. Application Data
(60) Provisional application No. 63/579,605, filed on Aug. 30, 2023.
(60) Provisional application No. 63/579,616, filed on Aug. 30, 2023.
(60) Provisional application No. 63/579,627, filed on Aug. 30, 2023.
(60) Provisional application No. 63/579,633, filed on Aug. 30, 2023.
(60) Provisional application No. 63/579,640, filed on Aug. 30, 2023.
(60) Provisional application No. 63/579,647, filed on Aug. 30, 2023.
(60) Provisional application No. 63/579,663, filed on Aug. 30, 2023.

(51) Int. Cl. H04R 25/00

(52) U.S. Cl. CPC H04R 25/606; H04R 2225/67

(57) ABSTRACT

Systems, methods, and devices include one or more acoustics-controlling device(s). The acoustics-controlling device(s) comprise implants, fixations, patches, and/or coatings formed of a metamaterial to create a particular behavior when exposed to sound waves. An acoustic metamaterial manipulates the acoustic waves that reach it. The metamaterial has a non-uniform material distribution, a non-uniform geometry, and/or a non-uniform material property, such as a non-uniform density, a non-uniform modulus of elasticity, a non-uniform bulk modulus, combinations thereof, and so forth. The system(s) include acoustic controlling patches and implants which control a path of an acoustic signal generated by one or more speakers. An acoustic-controlling patch can include an acoustic Fresnel lens or an acoustic Luneburg lens formed onto a substrate. Furthermore, manipulating the acoustic signal includes focusing the acoustic signal, forming an acoustic vortex from the acoustic signal, steering the acoustic signal, guiding the acoustic signal, or bending the acoustic signal.

### CROSS-REFERENCE TO RELATED APPLICATIONS

This application claims priority to U.S. Provisional Application Ser. No. 63/579,605 filed Aug. 30, 2023 and titled “FRUSTRATED TOTAL INTERNAL REFLECTION (FTIR) SURFACE TOPOGRAPHY AND COMPOSITION ANALYSIS SYSTEMS, METHODS, AND DEVICES;” U.S. Provisional Application Ser. No. 63/579,616 filed Aug. 30, 2023 and titled “SYSTEMS, METHODS, AND DEVICES OF WEARABLE ELECTRO-ACOUSTIC MONITORING;” U.S. Provisional Application Ser. No. 63/579,627 filed Aug. 30, 2023 and titled “SYSTEMS, METHODS, AND DEVICES FOR ACOUSTICALLY ENHANCING IMPLANTS;” U.S. Provisional Application Ser. No. 63/579,633 filed Aug. 30, 2023 and titled SYSTEMS, METHODS, AND DEVICES WITH SENSORS HAVING MULTIPLE DETECTION SIGNAL TYPES;” U.S. Provisional Application Ser. No. 63/579,640 filed Aug. 30, 2023 and titled MULTI-DEVICE HEALTH PARAMETER MONITORING SYSTEMS, METHODS, AND DEVICES;” U.S. Provisional Application Ser. No. 63/579,647 filed Aug. 30, 2023 and titled FRUSTRATED TOTAL INTERNAL REFLECTION (FTIR)-BASED HEALTH PARAMETER DETECTION SYSTEMS, METHODS, AND DEVICES;” and U.S. Provisional Application Ser. No. 63/579,663 filed Aug. 30, 2023 and titled “SYSTEMS, METHODS, AND DEVICES FOR NEUROLOGICAL AND/OR MUSCOSKELETAL PARAMETER CHARACTERIZATION;” the entireties of which are herein incorporated by reference.

### BACKGROUND

Medical acoustic implants are mostly used for hearing aid technology which amplify the audible sound. Delivering precise energy to specific body parts with high accuracy poses a significant challenge for devices that attempt to use acoustic waves for therapeutic purposes. These systems typically use sophisticated external acoustic generation techniques, which are complex, vary from patient to patient, and may carry the risk of potential damage. Other systems use a transducer as an implant which has its own technical difficulty in providing power to an implant.

It is with these observations in mind, among others, that various aspects of the present disclosure were conceived and developed.

### SUMMARY

Systems, methods, and devices can address the aforementioned issues. For instance an acoustic-enhancing system can include one or more speakers; and/or an acoustic-controlling patch formed of metamaterial having a non-uniform material distribution, a non-uniform geometry, or a non-uniform material property. The metamaterial can be operable to manipulate an acoustic signal from the one or more speakers directed into a body part of a user.

In some examples, the acoustic-controlling patch includes an acoustic Fresnel lens or an acoustic Luneburg lens formed onto a substrate. Also, manipulating the acoustic signal can include focusing the acoustic signal, forming an acoustic vortex from the acoustic signal, steering the acoustic signal, guiding the acoustic signal, or bending the acoustic signal. Additionally, the acoustic-enhancing system can include an acoustic-controlling implant including a bone graft patch with an array of acoustic focusing nodes; and/or one or more openings for placement around a transverse or spinous process. The wearable device can also be operable to provide at least one of an acoustic actuation or an acoustic sensing. Furthermore, the metamaterial can include at least one of a plurality of concentric circles of a first material disposed on a second material; a spiral of the first material disposed on the second material; and/or a plurality of pie slice-shaped sections with sequentially increasing percents of the first material in the second material.

In some instances, the acoustic-enhancing system can include a bone graft strip with a row of acoustic focusing nodes, the bone graft strip being operable to wrap around at least a portion of a bone. Moreover, the acoustic-enhancing system can include an acoustic-controlling spinal rod, the acoustic-controlling spinal rod having a circular cross-sectional profile. The non-uniform material distribution can include a concentric gradient with a minimum percent of acoustic-controlling material at an outer circumference of the circular cross-sectional profile and a maximum percent of the acoustic-controlling material at a center of the circular cross-sectional profile. Also, the acoustic-controlling patch can include an acoustic focusing node or an acoustic vortexing node, and/or the acoustic-controlling patch can have one or more screw receiving portions. The acoustic focusing node or the acoustic vortexing node can be operable to provide deep brain acoustic stimulation. Furthermore, the acoustic-enhancing system can further include a drug-eluting implant operable to release a drug responsive to a focused or vortexed acoustic signal resulting from a manipulation, by the acoustic-controlling patch, of the acoustic signal.

In some scenarios, an acoustic-controlling device can include a body having a primary medical function. The body can be formed of a metamaterial having a non-uniform material distribution of a one or more material such that the non-uniform material distribution is operable to controllably manipulate an acoustic signal from one or more speakers. The one or more material can form a three-dimensional implant including at least one of a sphere, a cube, or a cylinder. Additionally, the one or more material can include a first material being a coating on a second material, and/or the second material can include an implant. Furthermore, the acoustic-controlling device can be formed as a screw having a threaded portion. Also, the non-uniform material distribution can be operable to transmit the acoustic signal from the one or more speakers out a side of the screw to a fixation plate, and/or prevent transmission of the acoustic signal to the threaded portion. The metamaterial can include an acoustic impedance matching patch placed over a spinal rod to form an acoustic pathway through the spinal rod or around the spinal rod. Additionally, the non-uniform material distribution can be formed as at least one of a porous structure, a plurality of two-dimensional arrays, and/or a layered structure. The non-uniform material distribution can also cause the acoustic-controlling device to absorb the acoustic signal such that a target area is shielded from the acoustic signal by the acoustic-controlling device. Moreover, the target area shielded from the acoustic signal can include at least one of soft tissue, an organ, a nerve, or a bone.

In some examples, a method of controlling an acoustic signal includes emitting an acoustic signal from one or more speakers towards a body part of a user or an implant of the user; and/or manipulating the acoustic signal, using a metamaterial having a non-uniform material distribution, by at least one of focusing the acoustic signal; vortexing the acoustic signal; transmitting the acoustic signal; and/or absorbing the acoustic signal. Furthermore, manipulating the acoustic signal can include focusing the acoustic signal; and/or the method can further include eluting a drug from the implant of the user responsive to the implant receiving a focused acoustic signal.

### BRIEF DESCRIPTION OF THE DRAWINGS

The foregoing summary, as well as the following detailed description, will be better understood when read in conjunction with the appended drawings. For the purpose of illustration, there is shown in the drawings certain embodiments of the disclosed subject matter. It should be understood, however, that the disclosed subject matter is not limited to the precise embodiments and features shown. The accompanying drawings, which are incorporated in and constitute a part of this specification, illustrate an implementation of systems and methods consistent with the disclosed subject matter and, together with the description, serves to explain advantages and principles consistent with the disclosed subject matter, in which:

FIG. 1 illustrates an example system for controlling acoustic waves with one or more acoustic controlling devices.

FIG. 2 illustrates an example system for controlling acoustic waves with a bone graft patch.

FIGS. 3A-3C illustrate example systems for controlling acoustic waves with acoustic focusing patches.

FIGS. 4A and 4B illustrate example systems for controlling acoustic waves with acoustic focusing strips.

FIGS. 5A and 5B illustrates example systems for controlling acoustic waves with an acoustic spinal rod.

FIGS. 6A-6C illustrate example systems for controlling acoustic waves with a deep brain acoustic stimulation lens.

FIGS. 7A and 7B illustrate example systems for controlling acoustic waves with an acoustic lens and a drug-eluting implant.

FIGS. 7C and 7D illustrate example systems for controlling acoustic waves with an acoustic impedance implant and a drug-eluting implant.

FIG. 8 illustrates an example system for controlling acoustic waves with a three-dimensional acoustic lens.

FIGS. 9A-9C illustrate an example system for controlling acoustic waves with an acoustic-controlling implant coating or surface enhancement.

FIGS. 10-11B illustrate example systems for controlling acoustic waves with one or more acoustic-controlling screws.

FIGS. 12A-12D illustrate example systems for controlling acoustic waves with one or more acoustic impedance matching patches.

FIG. 13 illustrates example systems for controlling acoustic waves with one or more acoustic absorbing implants.

FIG. 14 illustrates an example method for controlling acoustic waves which can be performed by any of the systems depicted in FIGS. 1-13.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the presently disclosed technology or the appended claims. Further, it should be understood that any one of the features of the presently disclosed technology may be used separately or in combination with other features. Other systems, methods, features, and advantages of the presently disclosed technology will be, or become, apparent to one with skill in the art upon examination of the figures and the detailed description. It is intended that all such additional systems, methods, features, and advantages be included within this description, be within the scope of the presently disclosed technology, and be protected by the accompanying claims.

Further, as the presently disclosed technology is susceptible to embodiments of many different forms, it is intended that the present disclosure be considered as an example of the principles of the presently disclosed technology and not intended to limit the presently disclosed technology to the specific embodiments shown and described. Any one of the features of the presently disclosed technology may be used separately or in combination with any other feature. References to the terms “embodiment,” “example,” and/or the like in the description mean that the feature and/or features being referred to are included in, at least, one aspect of the description. Separate references to the terms “embodiment,” “examples,” and/or the like in the description do not necessarily refer to the same embodiment and are also not mutually exclusive unless so stated and/or except as will be readily apparent to those skilled in the art from the description. For instance, a feature, structure, process, step, action, or the like described in one embodiment may also be included in other embodiments but is not necessarily included. Thus, the presently disclosed technology may include a variety of combinations and/or integrations of the examples described herein. Additionally, all aspects of the present disclosure, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B and C.” An exception to this definition will occur only when a combination of elements, functions, steps or acts are in some way inherently mutually exclusive.

The systems, methods, and devices disclosed herein include acoustics-controlling devices such as implants, fixations, patches, and/or coatings, which exhibit a particular behavior when exposed to sound waves. An example of such a device is an object designed as an acoustic metamaterial that can manipulate the acoustic waves that reach it, for instance, to enhance cell growth, or for use in the treatment of tumors or diagnosis of infection, etc. The metamaterial can have a non-uniform material distribution, a non-uniform geometry, and/or a non-uniform material property (e.g., a non-uniform density, a non-unform modulus of elasticity, a non-uniform bulk modulus, combinations thereof, and so forth), The acoustic controlling patches and implants disclosed herein can provide the unique acoustic behavior with a simple procedure, and can reduce the risk of high intensity acoustic waves.

In some examples, the system includes an array of external speaker/transducers (e.g., a wearable device), which can generate initial acoustic waves. As the sound propagates through different predesigned implants, the acoustic wave can be manipulated for different medical purposes and/or health-related purposes. For instance, the acoustic wave can be focused on particular tissue (e.g., using a Fresnel lens or a Lumbung lens). Moreover, the sound wave can be spatially filtered, spiral sound-diffused, and/or shielded using a screw-shaped implant, as discussed in greater detail below.

In some scenarios, a group of acoustically enhanced wearables and/or implantable devices can include implants, coatings, fixations, patches, and so forth that have pre-designed structures with acoustic manipulation capabilities. These implants can be surgically or non-surgically placed inside or on the body using any applicable method, such as a suture, anchor, screws, bi-adhesive, wires, or staples. While these patches and implants show specific acoustic capabilities, they can have other specific, primary functionalities such as fixation.

The metamaterial can be pre-engineered to have a structure that transmits acoustic waves with a higher or lower propagation speed than the surrounding material (e.g., muscle tissue, water, etc.). The metamaterial can be formed of metals, polymers, ceramics, or so forth (e.g., aluminum, titanium, PEEK, PEK, PNMA, hydrogels, calcium phosphate, etc.) distributed onto the substrate in a particular arrangement to create the acoustic focusing/controlling effect. In some instances, the acoustics-controlling device can be formed of a biocompatible material, a biodegradable material, and/or a biological substance such as silk, or any various other materials. In some examples, air, or secondary materials can be selectively added to the material to control the sound propagation constant of the material, for instance, by lowering the speed at which sound passes through the material. Furthermore, the system disclosed herein can use various fixation techniques, such as an unaversive physical constraint, and/or an invasive physical constraint (e.g., a suture, an anchor, a screw, a bi-adhesive, a staple, and/or combinations thereof).

One or an array of speakers/transducers can generate acoustic wave at the surface of body. The generated sound wave can propagate through soft tissues, bone, and/or implants using the unusual acoustic properties of the device compared to natural material. Accordingly, when the sound wave passes through these devices, it is manipulated for specific purposes. The capabilities of these devices depends on their applications and can include acoustic focusing, spiral diffusion, spatial filtering, impedance matching, and shielding for therapeutic or diagnostic purposes.

Accordingly, the acoustic-controlling device can include different types of implant, such as a focusing implant, a transmission implant, and/or an absorbing implant. The focusing type of implant can include an acoustic spine bone graft patch; an acoustic patch; an acoustic spinal rod; a deep brain acoustic stimulation acoustic lens; an acoustic lensed drug-eluting implant; a 3D acoustic lens; implant coatings or surface enhancements; and/or combinations thereof. The transmission type of implant can include a screw with acoustic spatial filtering capability; an acoustic impedance matching patch for spinal rods and/or fixation plate; acoustic impedance patches for drug-eluting; and/or combinations thereof. The absorbing type of implant can include a dampening or absorbing implant to shield a region from the acoustic wave. Furthermore, the systems disclosed herein can include other wearable devices (e.g., leg sleeves, arm sleeves, gloves, cuffs, head caps, torso bands, back straps, and so forth) having acoustic actuators and/or acoustic sensors disposed thereon. For instance, the wearable device can include an array of acoustic sensors and/or actuators, which can operate with the speakers in conjunction with the presently disclosed acoustic controlling devices.

Additional advantages of the systems, methods, and devices discussed herein will become apparent from the detailed description below.

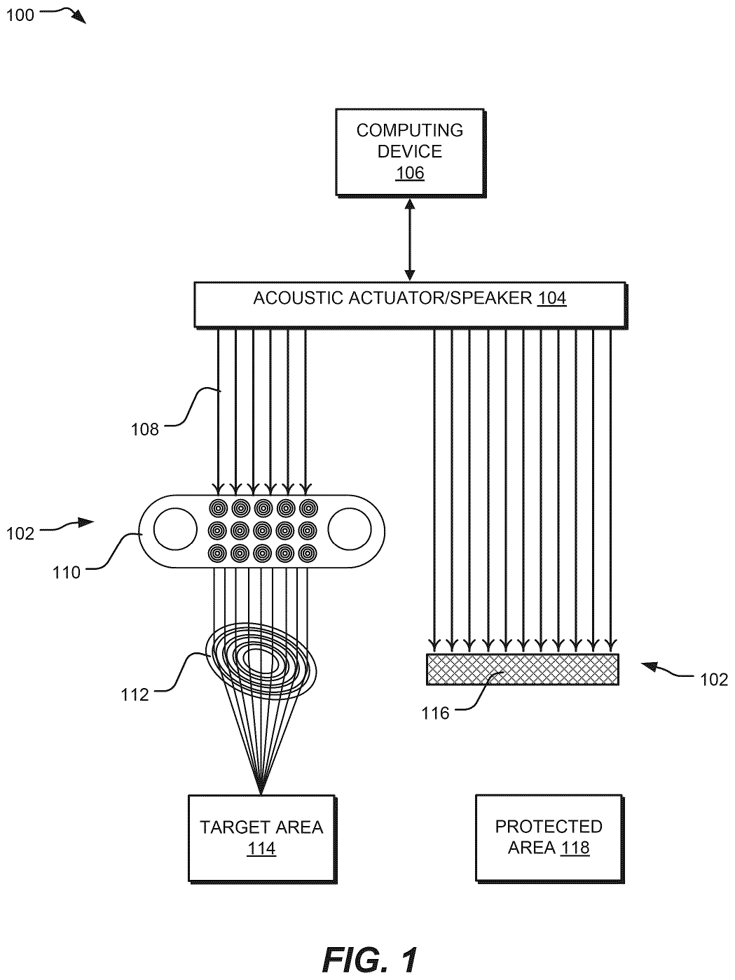

FIG. 1 illustrates an example system 100 for controlling acoustic waves with one or more acoustic controlling devices 102. The one or more acoustic controlling device 102 can control (e.g., focus, modify, redirect, transmit, absorb, etc.) an acoustic signal 108 (e.g., sound wave) generated by one or more speakers 104. The system 100 can include one or more computing system(s) 106, such as a microcontroller, processor, and/or memory. The memory can store computer-readable instructions that, when executed by the processor, cause the one or more speakers 104 to emit the acoustic signal 108 (e.g., an initial acoustic signal). In some instances, the one or more speakers 104 can be configured to emit a particular frequency or soundwave profile that corresponds to the type of acoustic controlling device 102 and/or the type of tissue or feature being targeted. For instance, the one or more speakers 104 can emit a higher frequency acoustic signal (e.g., 1 MHz-100 MHz) to stimulate cells for cell growth, and/or a lower frequency acoustic signal (e.g., 1 Hz-1 KHz) for large tissue stimulation, such as brain stimulation. A frequency of the acoustic signal 108 can also correspond to a tissue depth and/or a target distance of a focal point from the acoustic controlling device 102.

In some instances, an initial acoustic signal 108 can be transmitted from the one or more speakers 104 to the acoustic controlling device 102. By way of example the acoustic controlling device 102 can be an acoustic focusing patch 110 to focus, using one or more focusing nodes 112, the acoustic signal 108 to a target area 114. The system 100 can include various other acoustic controlling devices 102, additionally or alternatively to the acoustic focusing patch 110, such as an acoustic shield 116 for preventing the acoustic signal 108 from reaching a protected area 118. Additional examples of acoustic controlling device 102 are discussed in greater detail below.

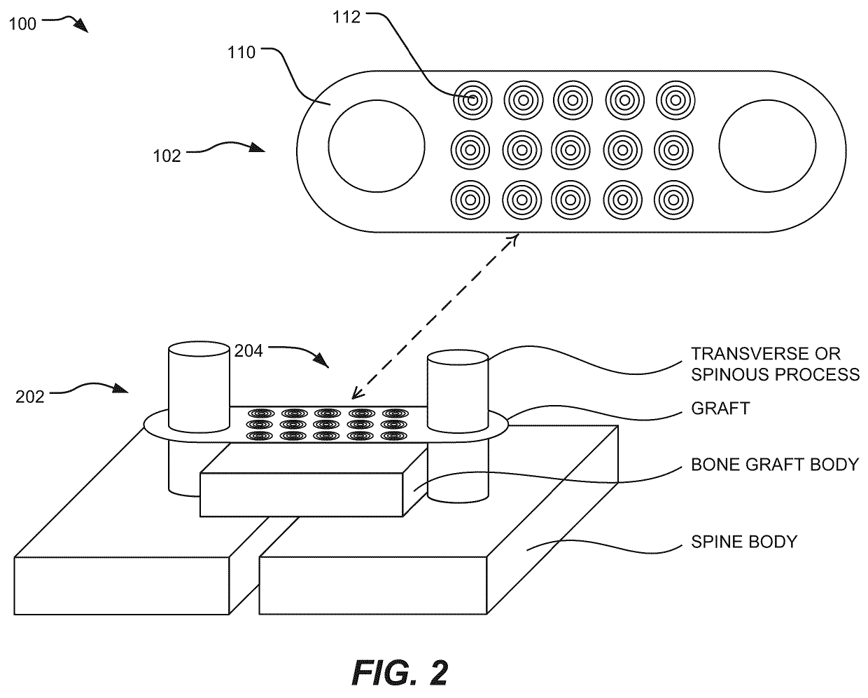

FIGS. 2-6C depict example systems 100 using one or more acoustic-controlling devices 102 to focus the acoustic signal 108. For instance, FIG. 2 depicts an example bone graft patch 202, such as an acoustic spine bone graft patch 204. The bone graft 202 patch can amplify the acoustic signal 108 generated by the external speaker 104 and focus it into the bone graft. This can improve healing in the bone graft. For example, the system 100 can include a graft patch having an array of acoustic focusing nodes 112 (e.g., circular conical focusers) disposed on a graft body. The bone graft patch can have one or more openings for placement around the transverse or spinous process to position the bone graft patch adjacent (e.g., abutting) and/or in close proximity to the bone graft body (e.g., spaced 0.1 mm-5 mm apart from the bone graft body). The bone graft body can be attached to and/or formed into a spine body.

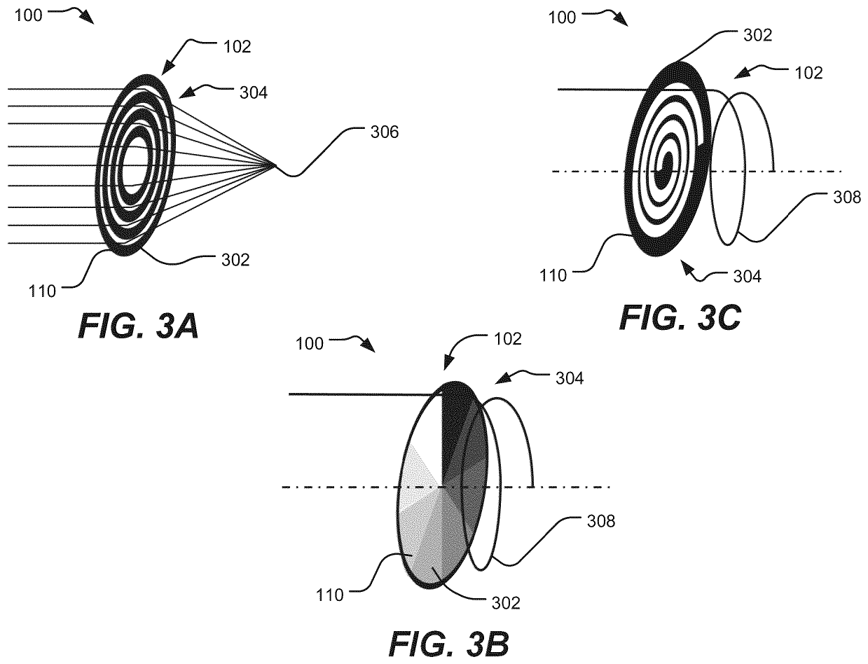

FIGS. 3A-3C depict different examples of one or more acoustic focusing patch(es) 110. The acoustic focusing patch(es) 110 can comprise a membrane 302 with a distribution 304 of acoustic transparence and/or an array of acoustic metamaterial capable of manipulating an acoustic field in a particular way corresponding to the distribution of acoustic transparence. The external speaker 104 can generate the initial acoustic wave 108. As the sound propagates through the array of metamaterials forming the acoustic focusing patch(es) 110, the acoustic wave can be focused on a target location 306 (e.g., as shown in FIG. 3A), and/or the acoustic focusing patch 110 can generate an acoustic vortex 308 (e.g., as shown in FIGS. 3B and 3C). The acoustic vortex 308 can channel the acoustic energy into a circular profile while omitting acoustic energy from the middle of the circle. The membrane material can fix the location of metamaterials to the body of the user (e.g., to their skin), and can have other application like fixing tissues.

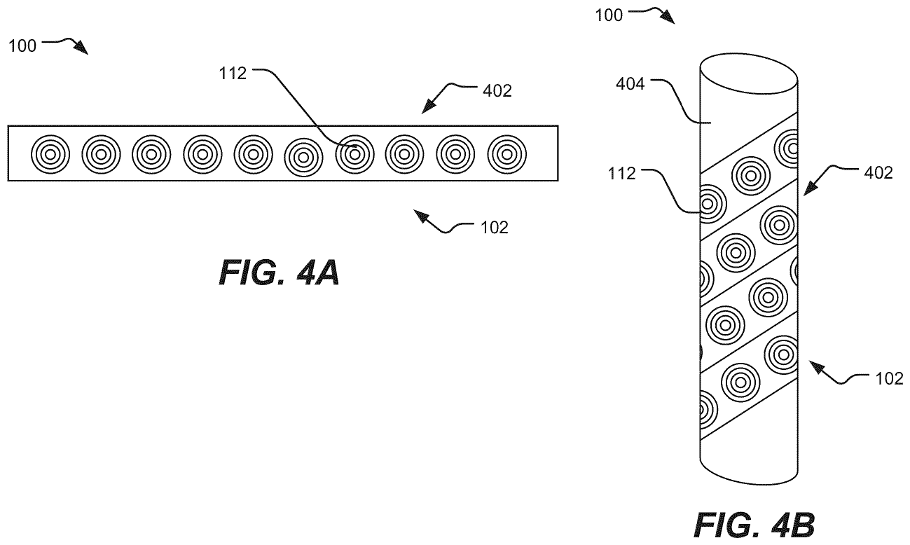

FIGS. 4A and 4B depict examples of one or more acoustic focusing strips 402 for acoustically manipulating bone by focusing and/or amplifying the sound signal generated by the speaker 104 into the bone 404. The acoustic bone focusing patch(es) can include a plurality of acoustic focusing nodes 112 disposed in one more rows. The acoustic focusing nodes 112 can form a single row, for instance, on a strip of substrate material. The strip of substrate material can be wrapped around a portion of a bone 404 to cover the portion of bone with the plurality of acoustic focusing nodes 112.

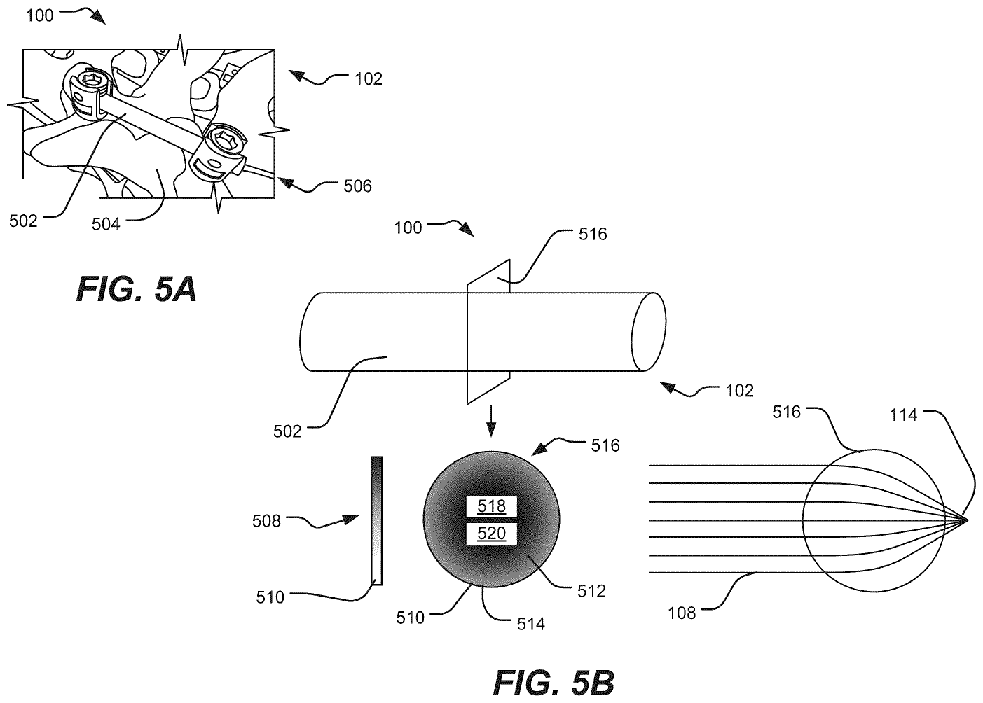

FIGS. 5A and 5B depict examples of one or more acoustic spinal rods 502, which can stabilize a spine segment 504 or other bone segment that is being fused, for instance, by holding the vertebrae together. Acoustic waves can be used to improve the healing process of bone grafts adjacent the spinal rod 502. In this way, an implant 506 having a body with a primary medical function (e.g., spinal support), can be formed of or modified to include a metamaterial having the acoustic controlling properties. The spinal rod 502 with acoustic capability can stabilize the spine and/or improve the healing process of the part of graft that in the acoustic shadow of the rod (e.g., abutting and/or adjacent the acoustic spinal rod). For instance, the acoustic spinal rod 502 can include a concentric gradient 508 with a minimum percent 510 of acoustic-controlling material 512 at an outer circumference 514 of the circular cross-sectional profile 516 and a maximum percent 518 of the acoustic-controlling material 512 at a center 520 of the circular cross-sectional profile 516. This gradient 508 can focus the initial acoustic wave 108 towards the target area 114.

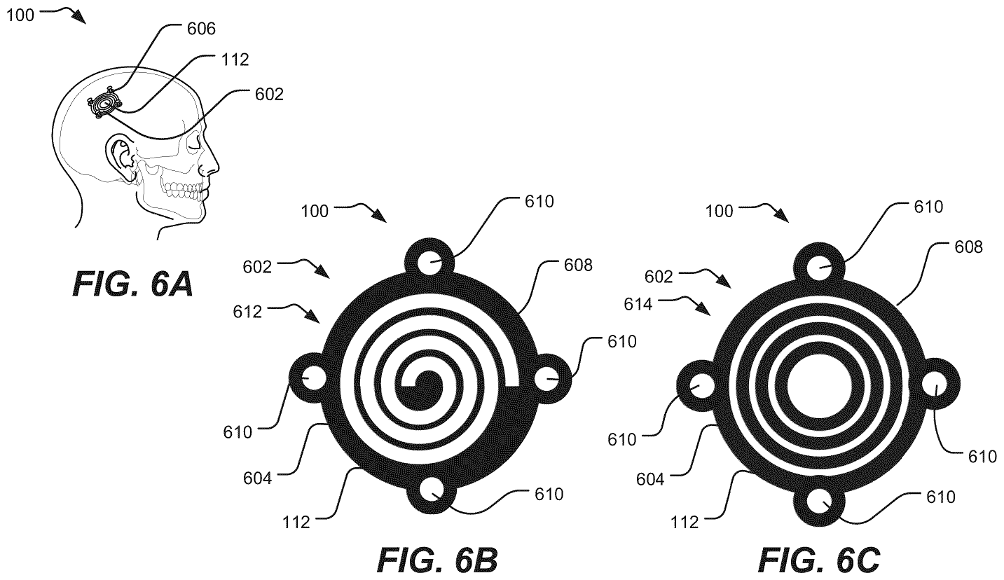

FIGS. 6A-6C depict examples of one or more deep brain acoustic stimulation acoustic lenses 602 (e.g., acoustic focusing nodes 112). For instance, the acoustic metamaterial 604 with focusing capability or spiral diffusing capability (e.g., as depicted in FIGS. 3A-3C) can be designed for and fixated to the skull using screws 606. For instance, the membrane, substrate, or body of the acoustic patch 608 can have one or more (e.g., two, three, four, five, or six) screw receiving portions 610. In some scenarios, with these implants, deep brain acoustic stimulation can be done without professional supervision and with help of an acoustic wearable. The acoustic patches 608 can include a spiral material distribution 612 and/or a concentric circle material distribution 614.

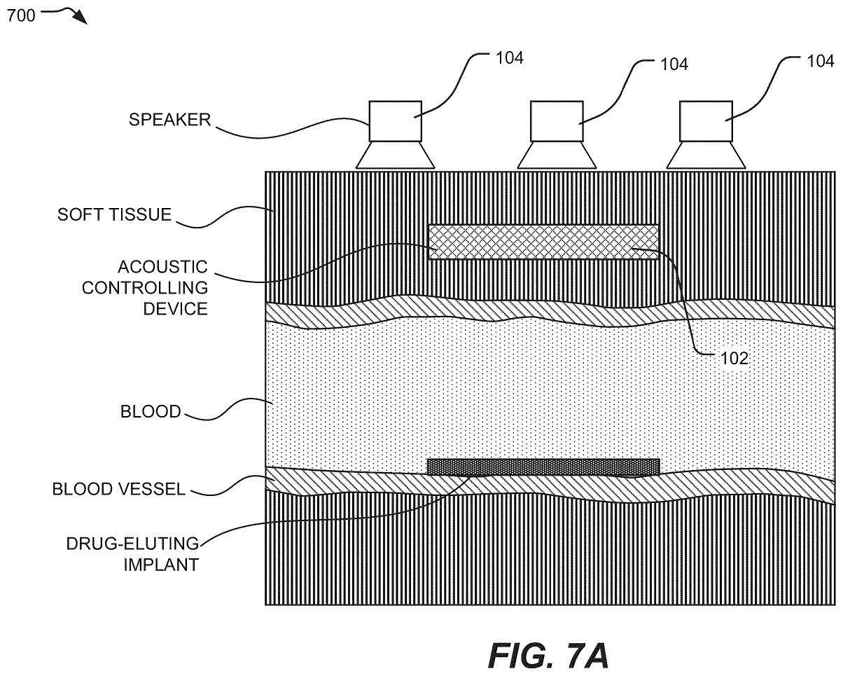

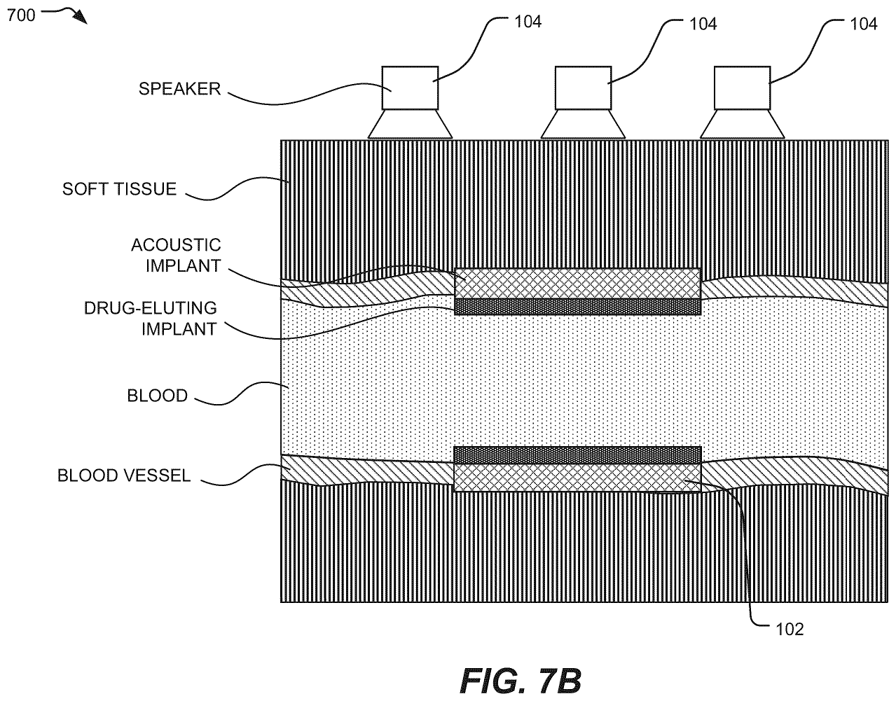

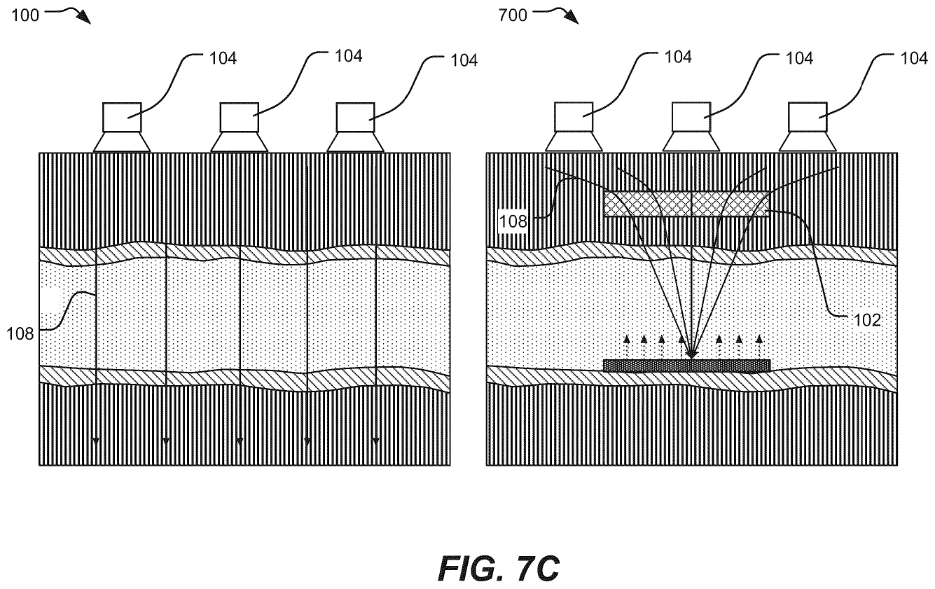

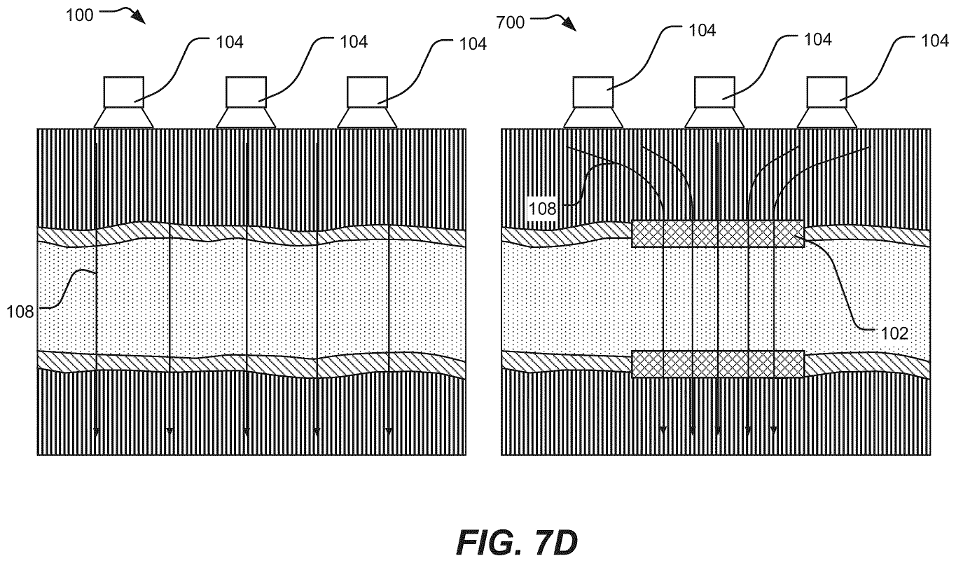

FIGS. 7A and 7B depict an example acoustic lensed drug-eluting system 700 including at least two parts: an acoustic lens (e.g., the acoustic controlling device 102) and a drug-eluting implant. The acoustic lensed drug-eluting system 700 can also include any other components discussed herein regarding the system 100. The drug-eluting implant can be an implant capable of releasing a particular dosage of a drug in the blood vessel or other soft or hard tissues. The dosage can be controlled by the kinetic energy of the acoustic waves impacting the drug-eluting implant. The acoustic lens can be a predesign structure that focuses the acoustic wave generated by the external source onto the drug eluting implant. As such, distribution of the drug from the drug eluting implant into the blood stream can be controlled by selectively activating (e.g., providing power) to the speaker 104 or other acoustic signal source. FIG. 7B depicts a first path of the acoustic signal 108 omitting the acoustic lens and/or the drug-eluting implant; and a second path of the acoustic signal 108 in a scenario including the acoustic lens and the drug-eluting implant. In the second path, the acoustic signal 108 is diverted/focused into the drug-eluting implant, causing it to release the drug. FIGS. 7C and 7D depict acoustic impedance patches for drug-eluting. For instance, the implants can absorb acoustic energy from the surrounding environment. This energy can increase the release rate of a drug with particular functionality. Furthermore, multiple metamaterials may be used at the same time. A drug eluting system 700 can include a first metamaterial of an acoustic controlling lens placed between the speaker 104, and/or a second metamaterial implanted in the body aligned with or integral with the drug-eluting implant. Additionally, a speaker 104 can be used in combination with another metamaterial, for instance, by placing another metamaterial on a transmission surface of the speaker 104, thus focusing or vortex the waves emitted from the speaker 104. By way of example, the drug-eluting implant can include a chemotherapeutic drug, which can be formed into a stint used to treat pancreatic cancer.

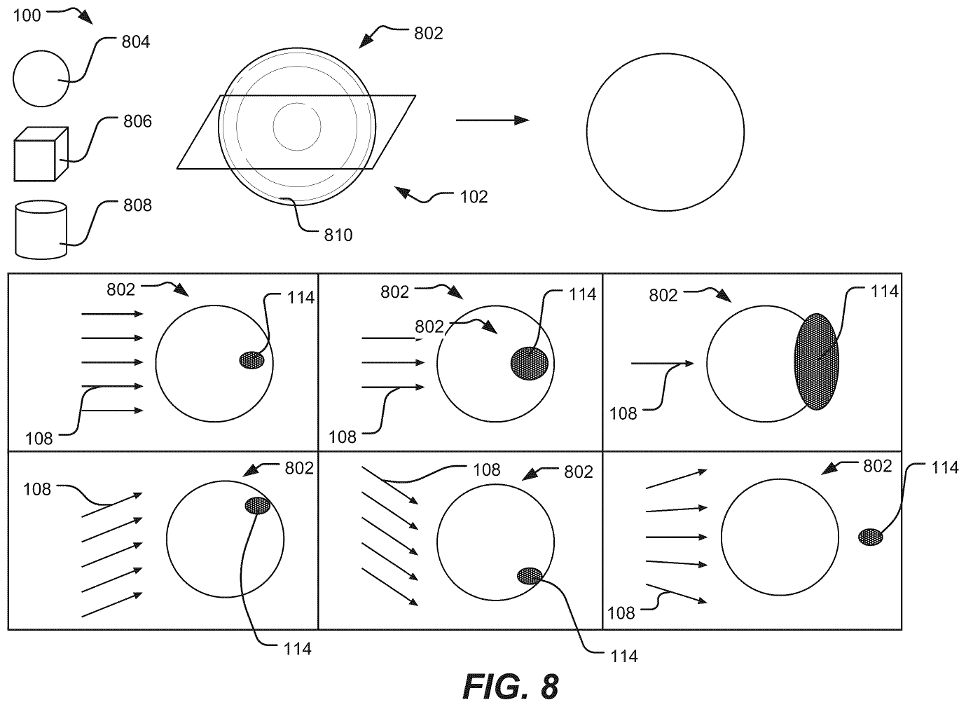

FIG. 8 depicts example 3D acoustic lenses 802 (e.g., spherical 804, cubical 806 or cylindrical 808 structure(s)) which can have a gradient acoustic characteristic. Sound with different frequencies and/or different angles of incidence can be focused at different location inside or outside of the 3D acoustic lens structure. By periodically changing frequency, directivity or incidence angle of the external acoustic wave 108, one or more particular target area(s) 114 can have the acoustic signal 108 focused upon it, generating acoustic stimulation at the target area(s) 114. These 3D lenses can be used as a stand-alone implant or can be used as part of a more complex implant system 100, for instance, using any combination or variety of acoustic-controlling devices 102 discussed herein. Furthermore, an implant coating 810 of metamaterial can be applied to the surface of the 3D lens 802, further enhancing the acoustic focusing capability. The surface coating 810 can be applied directly onto the surface and/or on a membrane as depicted in FIGS. 3A-3C. As such, the 3D implant can use the internal gradient 508 and/or the external coating 810 to control the acoustic signal 108 to increase blood flow and improve the healing process.

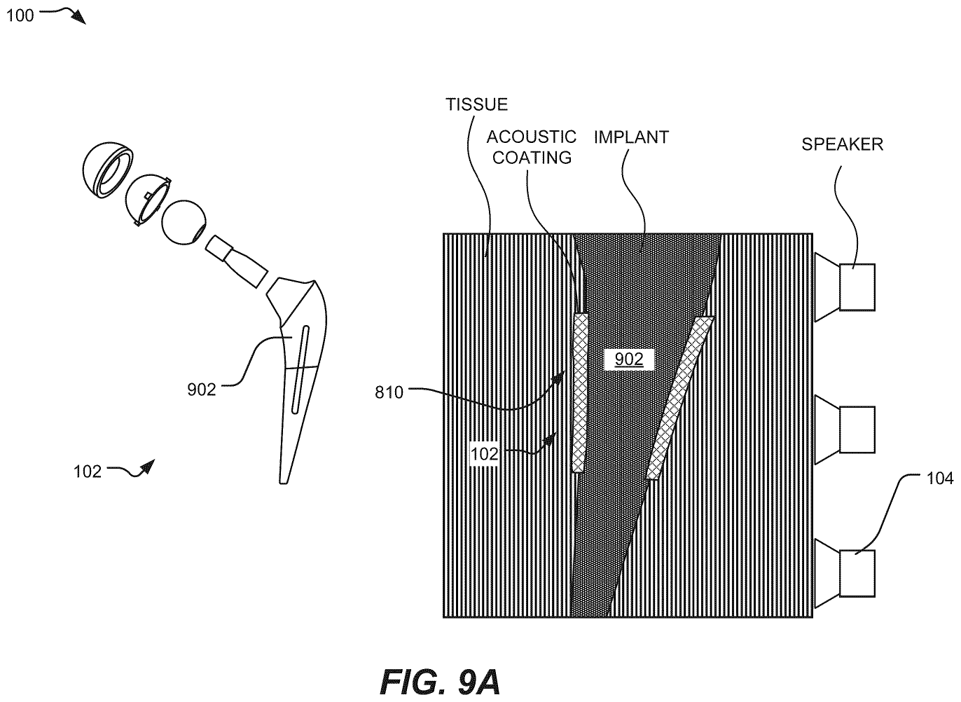

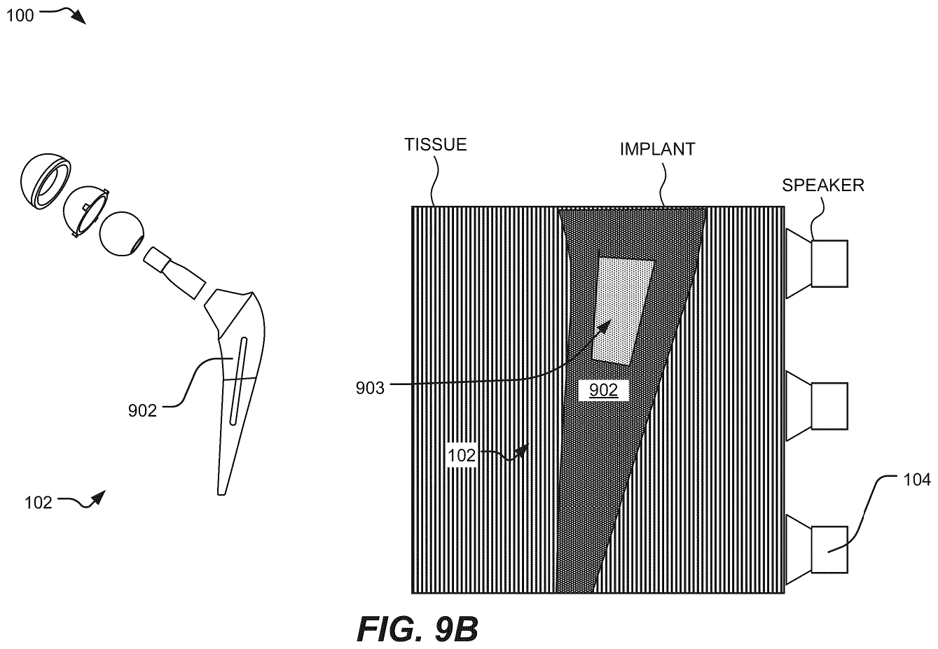

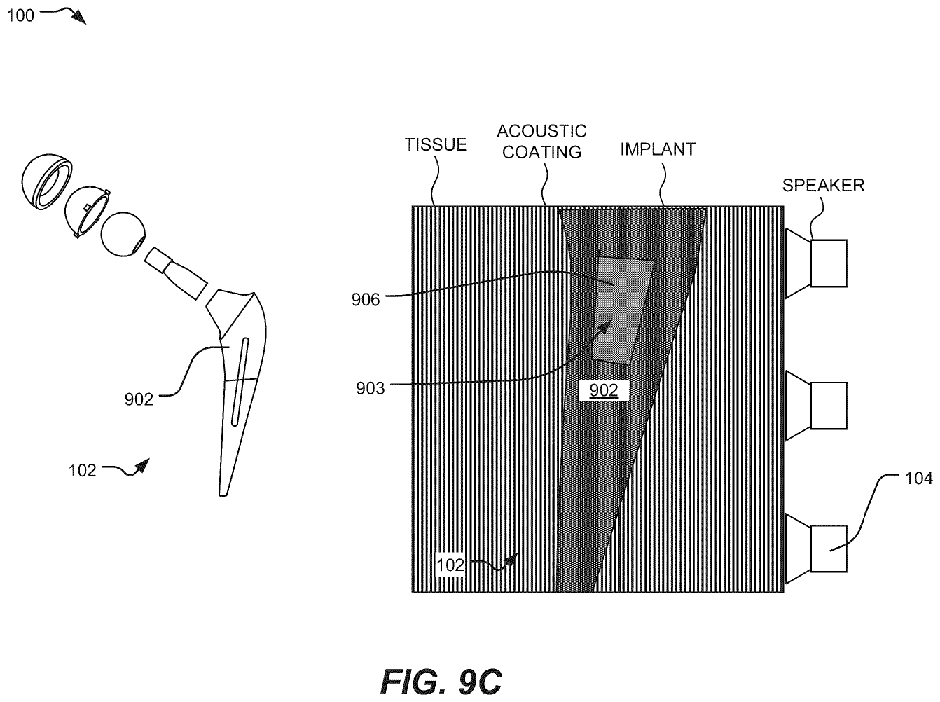

FIGS. 9A-9C depict an example of an acoustic-controlling implant coating 810 or surface enhancement 812. The coating can be a part of implant that plays the role of acoustic impedance matching and amplification at the same time. The implant coating 810 can be a different material than the implant 902 itself and can focus the acoustic energy at the surface of the implant to improve the cell attachment and healing process. The coating 810 or surface enhancement can be applied to an implant 902 having another primary function, such as a structural support function, a fixation function, a bracing function, or so forth. Furthermore, as shown in FIG. 9B, the implant 902 can have an inner, acoustic-controlling portion 903 that is of a similar or same material as the implant itself 902. For instance, the acoustic-controlling portion 903 of the implant 902 can be a structural modification inside the implant 902, such as a geometrical design formed into the material of the implant 902, a porous formation, a cavity, a change in density, or so forth. Additionally, geometrical structure can be formed into a surface of the implant 902. FIG. 9C depicts an example implant 902 having a portion 904 formed of a different material 906 than the implant 902 itself, thus forming an acoustic-controlling portion 908 of the implant 902 via a first material disposed inside a second material.

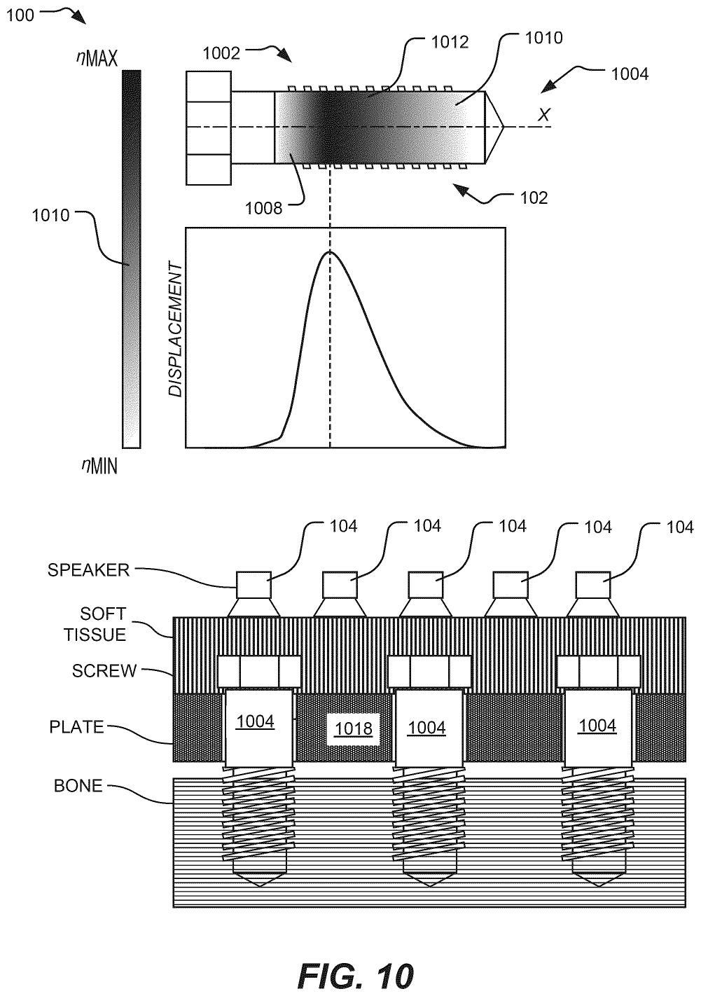

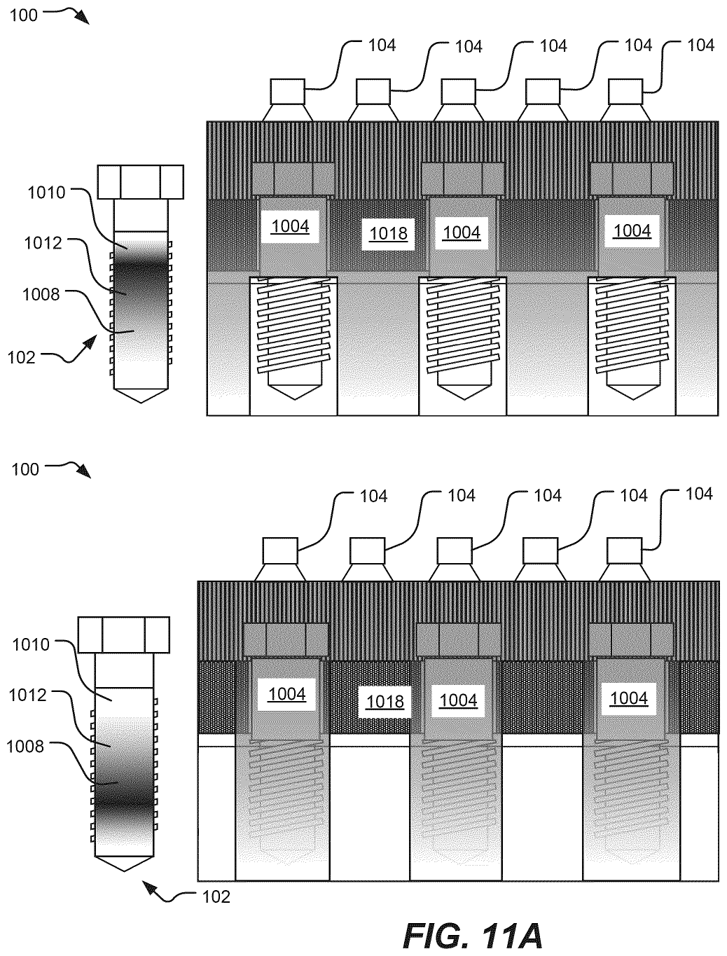

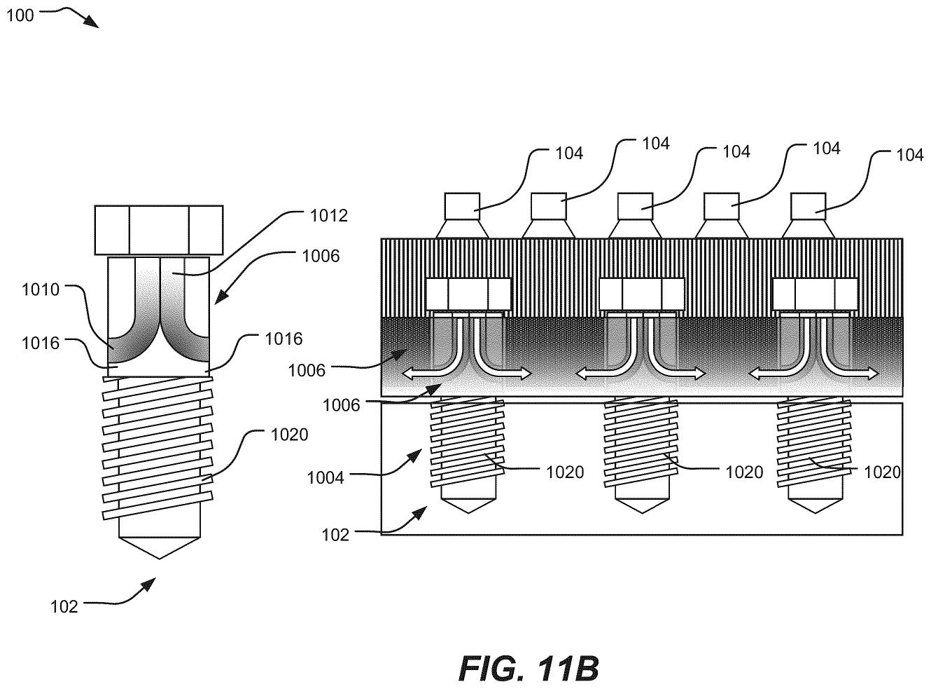

FIGS. 10-13 depict example systems 100 using acoustic-controlling devices 102 including one or more transmission implants 1002. FIGS. 10-11B depict an acoustic-controlling screw 1004 for acoustic spatial filtering 1006. The acoustic-controlling screw 1004 can have an interior structure 1008 including a metamaterial gradient 1010 to define the acoustic-controlling effect. For instance, the screw 1004 can have a particular acoustic material gradient profile 1012 that transmits acoustic energy 1014 out a side 1016 of the screw 1004 to the internal fixation plate 1018. As such, the internal fixation plate 1018 can vibrate for improving the healing process while omitting transmission of the acoustic energy to the threads 1020, which can prevent loosening caused by the vibration. The acoustic-controlling screw 1004 can have a primary function as a structural screw for securing the fixation plate 1018 in place, or the acoustic-controlling screw 1004 can exclusively operate to transmit the acoustic signal 108 to the fixation plate 1018 while omitting any primary securing/fixation functionality. In some scenarios, the plate vibrations caused by the acoustic signal 108 can stimulate bone growth into the plate 1018. In situations including an asymmetrical plate and/or asymmetrical tissue, vibrating a portion of the plate 1018 can compensate for the asymmetry and cause more even bone growth on the plate than would otherwise occur. Furthermore, as shown in FIG. 11B, an acoustic transmission pathway 1022 can be defined by the metamaterial 1024 in the screw 1004, having a high-to-low material gradient 1010 or density channel or vice versa, to direct the acoustic waves to a side 1014 of the screw 1004 or in any particular direction.

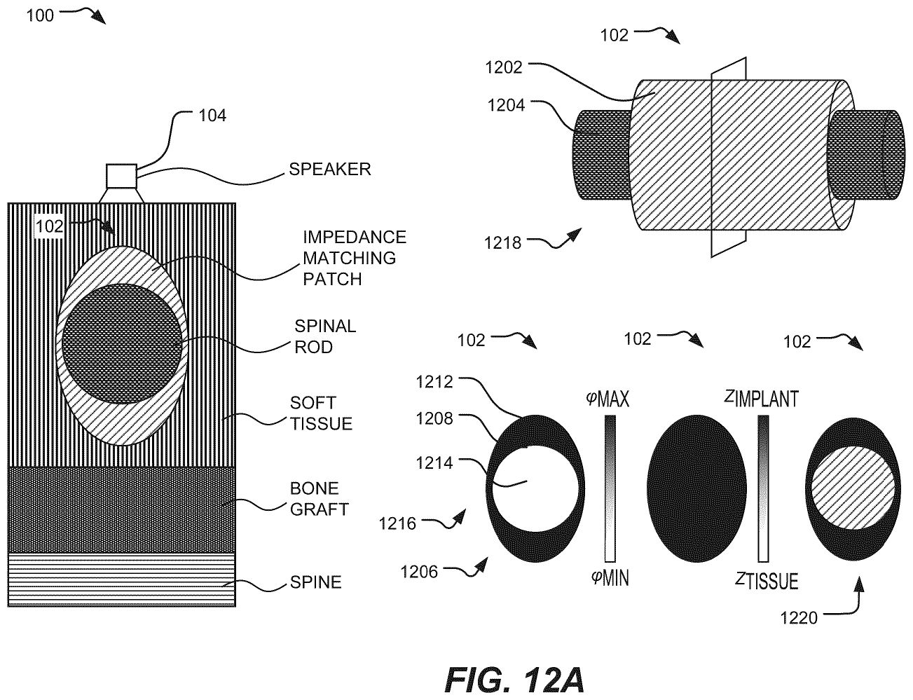

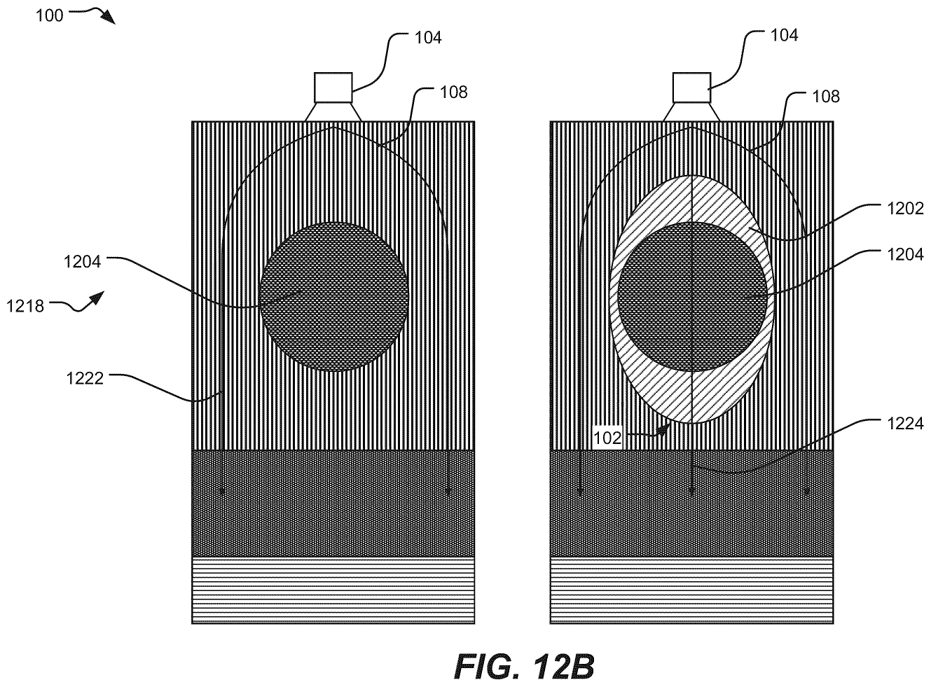

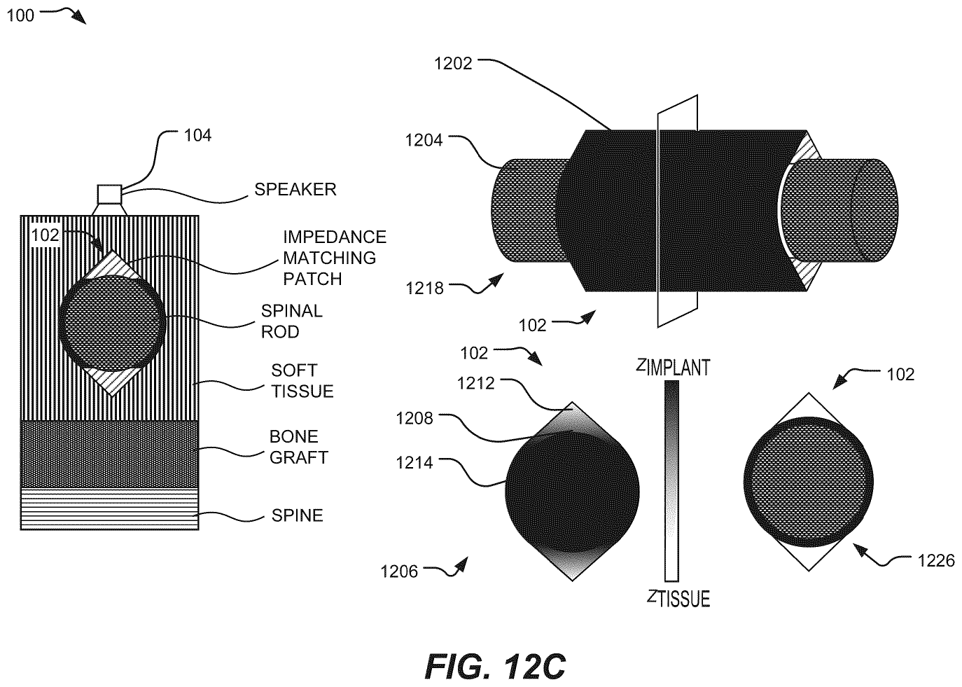

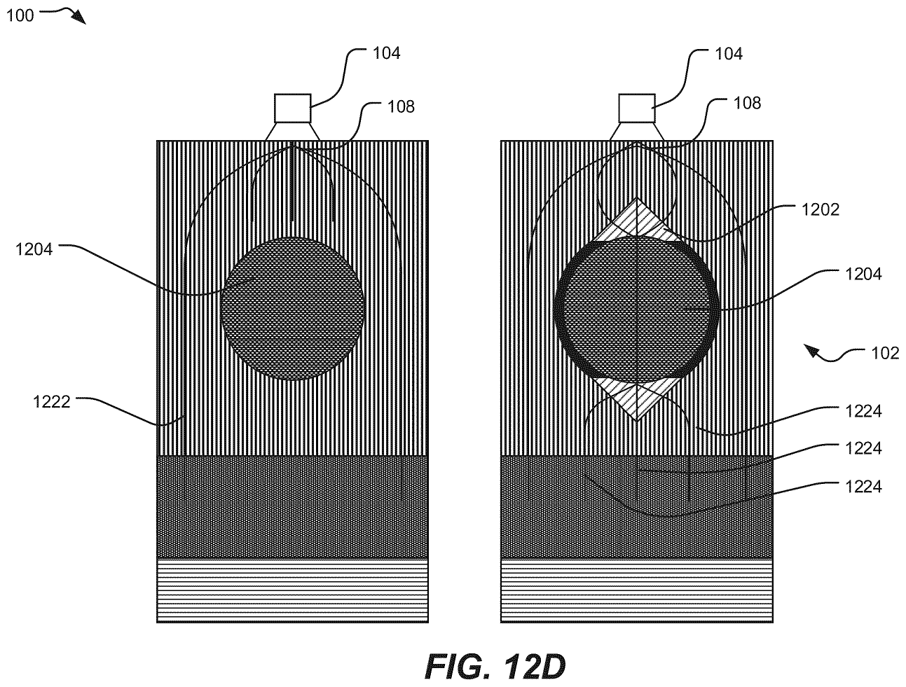

FIGS. 12A-12D depict example systems 100 including acoustic impedance matching patches 1202 for spinal rods 1204 and/or fixation plates. These types of acoustic-controlling devices 102 can include a patch 1202 installed on the existing spinal rods 1204, for instance, as a retrofit. Additionally or alternatively, the patches 1202 can be used on the acoustic controlling rods 502 discussed above. The patches 1202 can have a gradient reflective index 1206 (impedance) located in the boundary 1208 of two mediums with significantly different acoustic impedance. In these patches, the acoustic impedance (e.g., speed of sound) can gradually change from medium #1 1210 to medium #2 1214. This gradual change 1216 can reduce the reflection of acoustic wave and improve the transition of acoustic waves.

As shown in FIG. 12A, the implant 1218 can have two matching layers, both in the propagation pattern of sound. One layer can be an outer layer near or adjacent the skin, and the other layer can be an inner layer near or adjacent the rod 1204. The patch material can be biodegradable (silk) to dissolve over time.

Furthermore, the acoustic impedance matching implant 1218 can have one or more of three structures: a porous structure, a two-dimensional array, and/or a layered structure. FIG. 12A depicts a first example of a porous structure implant 1220 and a two-dimensional array structure implant. The porous structure 1220 can be manufactured using a chemical process and/or using 3D printing. As such, the porosity can be carefully controlled to create a particular acoustic-controlling effect. Moreover, the porosity can correspond to a particular size to promote cell growth into the pores. FIG. 12B shows a first acoustic pathway 1222 of the acoustic signal 108 onto the spinal rod in a scenario omitting the patch. FIG. 12B also shows a second acoustic pathway 1224 of the acoustic signal 108 passing through/around the rod in scenarios with the acoustic implant. FIG. 12C depicts an example layered structure 1226 of the acoustic controlling patch. FIG. 12D shows a first acoustic pathway of the acoustic signal 108 onto the spinal rod in a scenario omitting the layered structure patch. FIG. 12D also shows a second acoustic pathway 1224 of the acoustic signal 108 passing through/around the rod 1204 in scenarios with the layered structure 1226 acoustic implant. The rod 1204 (e.g., and any of the acoustic-controlling devices 102 discussed herein) can be used to manipulate shear acoustic waves and/or pressure acoustic waves.

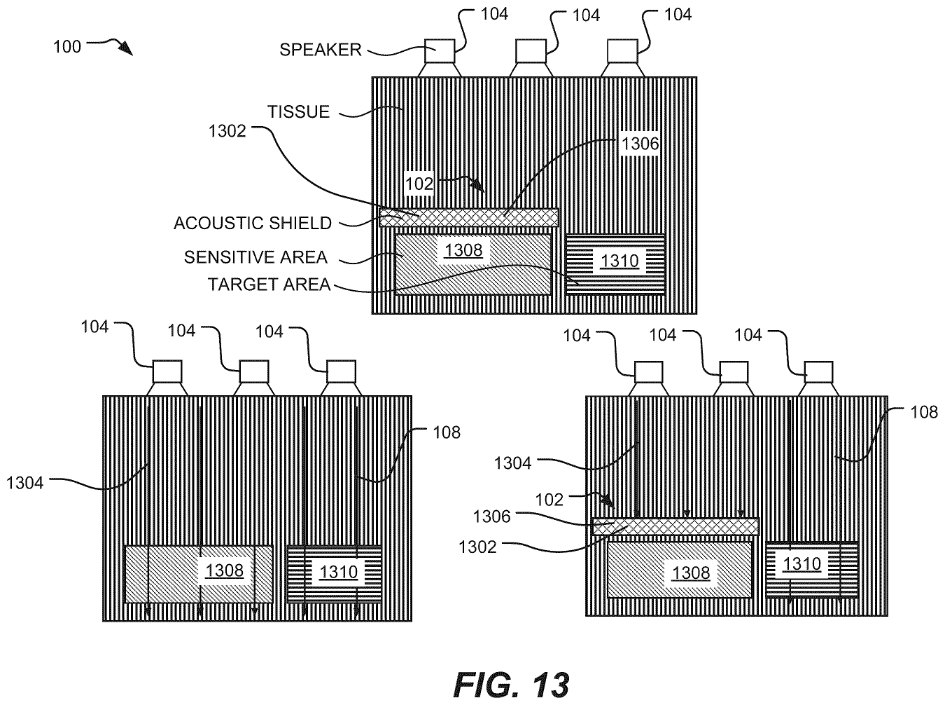

FIG. 13 depicts an example acoustic absorbing implant 1302 for absorbing high-energy acoustic waves 1304 that may have potential to cause temporary or prominent damage to various body parts such as organs, soft tissues, nerves, or bones. The damage can depend on factors such as the intensity, frequency, duration of the acoustic signal 108. There are two primary ways in which acoustic waves can cause damage: direct mechanical effects and/or cavitation. Using metamaterials 1306 to shield the surrounding tissue can be beneficial in medical cases where one desires to use the therapeutic effects of acoustic waves while minimizing potential damage to the adjacent tissue. The metamaterial 1306 can shield sensitive areas 1308 by two mechanisms: damping the acoustic energy and/or reflecting the acoustic energy to unsensitive areas and/or the target area 1310.

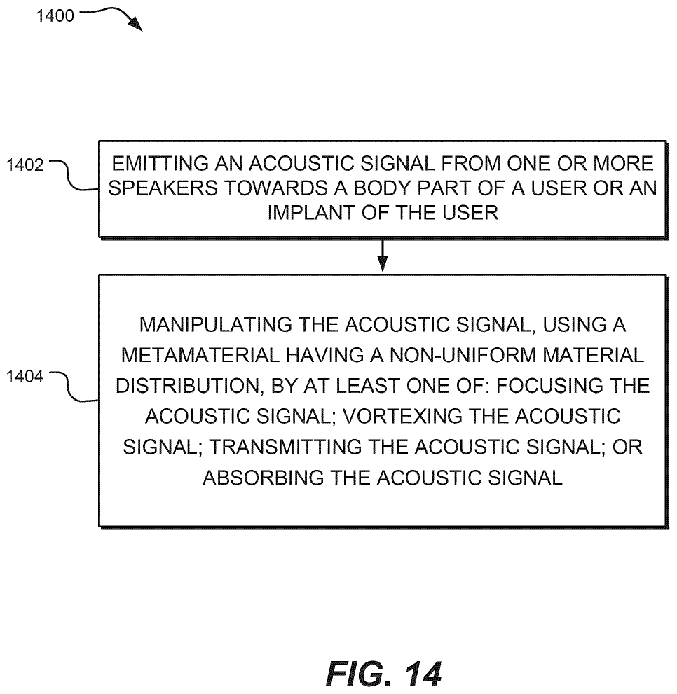

FIG. 14 depicts an example method of controlling an acoustic signal 108 with an acoustic-controlling device 102.

In some examples, at operation 1402, the method 1400 can emit an acoustic signal from one or more speakers towards a body part of a user or an implant of the user. At operation 1404, the method 1400 can manipulate the acoustic signal, using a metamaterial having a non-uniform material distribution, by at least one of: focusing the acoustic signal; vortexing the acoustic signal; transmitting the acoustic signal; or absorbing the acoustic signal; or steering, guiding, or bending the waves.

It is to be understood that the specific order or hierarchy of steps in the method(s) depicted throughout this disclosure are instances of example approaches and can be rearranged while remaining within the disclosed subject matter. For instance, any of the operations depicted throughout this disclosure may be omitted, repeated, performed in parallel, performed in a different order, and/or combined with any other of the operations depicted throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. An acoustic-enhancing system comprising:
one or more speakers; and
an acoustic-controlling patch formed of metamaterial having a non-uniform material distribution, a non-uniform geometry, or a non-uniform material property, the metamaterial is operable to manipulate an acoustic signal from the one or more speakers directed into a body part of a user.

2. The acoustic-enhancing system of claim 1,
wherein,
the acoustic-controlling patch includes an acoustic Fresnel lens or an acoustic Luneburg lens formed onto a substrate; and
manipulating the acoustic signal includes focusing the acoustic signal, forming an acoustic vortex from the acoustic signal, steering the acoustic signal, guiding the acoustic signal, or bending the acoustic signal.

3. The acoustic-enhancing system of claim 2, further comprising:
an acoustic-controlling implant including a bone graft patch with:
an array of acoustic focusing nodes; or
one or more openings for placement around a transverse or spinous process.

4. The acoustic-enhancing system of claim 3, further comprising:
a wearable device operable to provide at least one of an acoustic actuation or an acoustic sensing.

5. The acoustic-enhancing system of claim 1,
wherein,
the metamaterial includes at least one of:
a plurality of concentric circles of a first material disposed on a second material;
a spiral of the first material disposed on the second material; or
a plurality of pie slice-shaped sections with sequentially increasing percents of the first material in the second material.

6. The acoustic-enhancing system of claim 1, further comprising:
a bone graft strip with a row of acoustic focusing nodes, the bone graft strip being operable to wrap around at least a portion of a bone.

7. The acoustic-enhancing system of claim 1, further comprising:
an acoustic-controlling spinal rod, the acoustic-controlling spinal rod having a circular cross-sectional profile; and
the non-uniform material distribution includes a concentric gradient with a minimum percent of acoustic-controlling material at an outer circumference of the circular cross-sectional profile and a maximum percent of the acoustic-controlling material at a center of the circular cross-sectional profile.

8. The acoustic-enhancing system of claim 1,
wherein,
the acoustic-controlling patch includes an acoustic focusing node or an acoustic vortexing node, the acoustic-controlling patch having one or more screw receiving portions.

9. The acoustic-enhancing system of claim 8,
wherein,
the acoustic focusing node or the acoustic vortexing node is operable to provide deep brain acoustic stimulation.

10. The acoustic-enhancing system of claim 1, further comprising:
a drug-eluting implant operable to release a drug responsive to a focused or vortexed acoustic signal resulting from a manipulation, by the acoustic-controlling patch, of the acoustic signal.

11. An acoustic-controlling device comprising:
a body having a primary medical function, the body formed of a metamaterial having a non-uniform material distribution of a one or more material such that the non-uniform material distribution is operable to controllably manipulate an acoustic signal from one or more speakers.

12. The acoustic-controlling device of claim 11,
wherein,
the one or more material forms a three-dimensional implant including at least one of a sphere, a cube, or a cylinder.

13. The acoustic-controlling device of claim 11,
wherein,
the one or more material includes a first material is a coating on a second material; and
the second material includes an implant.

14. The acoustic-controlling device of claim 11,
wherein,
the acoustic-controlling device is formed as a screw having a threaded portion; and
the non-uniform material distribution is operable to:
transmit the acoustic signal from the one or more speakers out a side of the screw to a fixation plate, and
prevent transmission of the acoustic signal to the threaded portion.

15. The acoustic-controlling device of claim 11,
wherein,
the metamaterial includes an acoustic impedance matching patch placed over a spinal rod to form an acoustic pathway through the spinal rod or around the spinal rod.

16. The acoustic-controlling device of claim 11,
wherein,
the non-uniform material distribution is formed as at least one of a porous structure, a plurality of two-dimensional arrays, or a layered structure.

17. The acoustic-controlling device of claim 11,
wherein,
the non-uniform material distribution causes the acoustic-controlling device to absorb the acoustic signal such that a target area is shielded from the acoustic signal by the acoustic-controlling device.

18. The acoustic-controlling device of claim 17,
wherein,
the target area shielded from the acoustic signal includes at least one of soft tissue, an organ, a nerve, or a bone.

19. A method of controlling an acoustic signal, the method comprising:
emitting an acoustic signal from one or more speakers towards a body part of a user or an implant of the user; and
manipulating the acoustic signal, using a metamaterial having a non-uniform material distribution, by at least one of:
focusing the acoustic signal;
vortexing the acoustic signal;
transmitting the acoustic signal; or
absorbing the acoustic signal.

20. The method of claim 19,
wherein,
manipulating the acoustic signal includes focusing the acoustic signal; and
the method further includes eluting a drug from the implant of the user responsive to the implant receiving a focused acoustic signal.
